(19) United States
(12) Patent Application Publication
(10) Pub. No.: US 2010/0178362 A1
(43) Pub. Date: Jul. 15, 2010

(54) CHROMIUM COMPLEXES FOR IMPROVEMENT OF MEMORY AND COGNITIVE FUNCTION

(76) Inventors: James R. Komorowski, Trumbell, CT (US); Vijaya Juturu, Dobbs Ferry, NY (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/652,282

(22) Filed: Jan. 5, 2010

Related U.S. Application Data
(63) Continuation of application No. PCT/US2008/069079, filed on Jul. 2, 2008.
(60) Provisional application No. 60/948,429, filed on Jul. 6, 2007.

(51) Int. Cl. A61K 33/24; A61K 36/06; A61K 31/555; A61P 25/00; A61P 25/28

(52) U.S. Cl. 424/655; 424/195.16; 514/184; 514/188

(57) ABSTRACT

A method of treating impaired memory and/or cognitive function by administering one or more chromium complexes, for example chromium picolinate, to an individual in need of improved memory and/or cognitive function. This method is used to improve memory and/or cognitive function associated with various disorders, including Alzheimer's disease, dementia, and mild cognitive impairment (MCI). Chromium complexes for treating memory or cognitive impairment are also disclosed.

### RELATED APPLICATIONS

The present application is a continuation of PCT/US2008/069079, filed Jul. 2, 2008, which designated the United States and was published in English, which claims priority under 35 U.S.C. §119(a)-(d) to U.S. Provisional Application Ser. No. 60/948,429, filed on Jul. 6, 2007, by Komorowski et al., entitled “CHROMIUM COMPLEXES FOR IMPROVEMENT OF MEMORY AND COGNITIVE FUNCTION.” The content of each of these applications is hereby incorporated by reference in its entirety.

### BACKGROUND OF THE INVENTION

1. Field of the Invention

The present invention relates to a method of improving memory and/or cognition by administering a chromium complex such as chromium picolinate to an individual with impaired memory and/or cognition, such as an individual having, for example, mild cognitive impairment, Alzheimer's disease or dementia.

2. Description of the Related Art

Impairment of memory and/or cognition occurs in numerous disorders, particularly aging, Alzheimer's Disease and dementia. Alzheimer's disease is a progressive brain disorder that gradually destroys a person's memory and ability to learn, reason, make judgments, communicate and carry out daily activities. As Alzheimer's progresses, individuals may also experience changes in personality and behavior, such as anxiety, suspiciousness or agitation, as well as delusions or hallucinations. There are now more than 5 million people in the United States living with Alzheimer's disease. This number includes 4.9 million people over the age of 65 and between 200,000 and 500,000 people under age 65 with early-onset Alzheimer's disease and other dementias. Dementia is defined as a deterioration of intellectual faculties, such as memory, concentration, and judgment, resulting from an organic disease or a disorder of the brain. It is sometimes accompanied by emotional disturbance and personality changes. Although much less significant than Alzheimer's, mild cognitive impairment is a common condition in the aging population, and few treatments for this condition exist.

None of the currently available treatments for loss of memory and cognition, including those associated with Alzheimer's Disease, are very effective. Thus, there is a continuous need for new therapeutics that will improve memory and cognition.

### SUMMARY OF THE INVENTION

In one embodiment, a method of treating a cognitive condition associated with reduced brain insulin levels is provided, the method comprising administering an effective brain insulin level-increasing amount of a chromium complex, or pharmaceutically acceptable salt thereof, to the mammal. In one embodiment, the reduced brain insulin levels are associated with impaired memory and/or cognition. In one aspect of this embodiment, the impaired memory and/or cognitive function is associated with Alzheimer's disease, dementia, mild cognitive impairment (MCI), amnesia or diabetes. In one embodiment, the chromium complex is chromium picolinate, chromic tripicolinate, chromium nicotinate, chromic polynicotinate, chromium chloride, chromium histidinate, chromium nicotinate-glycinate, chromium phenylalanine or chromium yeast. The method may further comprise administering a cholinesterase inhibitor, memanitine, vitamin E a phospholipid or an omega-3 fatty acid to the individual. In one embodiment, the mammal is a human.

In one embodiment, a method of treating impaired memory and/or cognitive function in a mammal in need thereof is provided, the method comprising identifying an individual with impaired memory and/or cognitive function; and administering an effective memory and/or cognitive function-enhancing amount of a chromium complex, or pharmaceutically acceptable salt thereof, to the mammal. In one embodiment, the impaired memory and/or cognitive function is associated with Alzheimer's disease, dementia, mild cognitive impairment (MCI), amnesia or diabetes. In another embodiment, the chromium complex is chromium picolinate, chromic tripicolinate, chromium nicotinate, chromic polynicotinate, chromium chloride, chromium histidinate, chromium nicotinate-glycinate, chromium phenylalanine or chromium yeast. The method may further comprise administering a cholinesterase inhibitor, memanitine, vitamin E, a phospholipid or an omega-3 fatty acid to the individual. In one embodiment, the mammal is a human.

In another embodiment, a method of preventing impaired memory and/or cognitive function in a mammal in need thereof is provided, the method comprising identifying a mammal in need of prevention of impaired memory and/or cognitive function; and administering an effective memory and/or cognitive function-enhancing amount of a chromium complex, or pharmaceutically acceptable salt thereof, to the mammal. In one embodiment, the chromium complex is chromium picolinate, chromic tripicolinate, chromium nicotinate, chromic polynicotinate, chromium chloride, chromium histidinate, chromium nicotinate-glycinate, chromium phenylalanine or chromium yeast. The method may further comprise administering a cholinesterase inhibitor, memanitine, vitamin E a phospholipid or an omega-3 fatty acid to the individual. In one embodiment, the mammal is a human.

In one embodiment, a method of treating a cognitive condition associated with abnormal brain glucose levels is provided, the method comprising identifying a mammal suffering from a cognitive condition associated with abnormal brain glucose levels; and administering an effective brain glucose level-normalizing amount of a chromium complex, or pharmaceutically acceptable salt thereof, to the mammal. In one embodiment, the abnormal brain glucose levels are associated with impaired memory and/or cognition. In one embodiment, the impaired memory and/or cognitive function is associated with Alzheimer's disease, dementia, mild cognitive impairment (MCI), amnesia or diabetes. In another embodiment, the chromium complex is chromium picolinate, chromic tripicolinate, chromium nicotinate, chromic polynicotinate, chromium chloride, chromium histidinate, chromium nicotinate-glycinate, chromium phenylalanine or chromium yeast. The method may further comprise administering a cholinesterase inhibitor, memanitine, vitamin E, a phospholipid or an omega-3 fatty acid to the individual. In one embodiment, the mammal is a human.

In one embodiment, a method of treating a cognitive condition associated with brain insulin resistance is provided, the method comprising identifying a mammal suffering from a cognitive condition associated with brain insulin resistance; and administering an effective brain glucose level-normalizing amount of a chromium complex, or pharmaceutically acceptable salt thereof, to the mammal. In one embodiment, the brain insulin resistance is associated with impaired memory and/or cognition. In one embodiment, the impaired memory and/or cognitive function is associated with Alzheimer's disease, dementia, mild cognitive impairment (MCI), amnesia or diabetes. In another embodiment, the chromium complex is chromium picolinate, chromic tripicolinate, chromium nicotinate, chromic polynicotinate, chromium chloride, chromium histidinate, chromium nicotinate-glycinate, chromium phenylalanine or chromium yeast. The method may further comprise administering a cholinesterase inhibitor, memanitine, vitamin E, a phospholipid or an omega-3 fatty acid to the individual. In one embodiment, the mammal is a human.

In one embodiment, a method for supporting cognitive function is provided, the method comprising providing a dietary chromium complex to the individual; advising the individual that the dietary chromium complex may support cognitive function; and administering or self-administering the dietary chromium complex to the individual. In another embodiment, the chromium complex is chromium picolinate, chromic tripicolinate, chromium nicotinate, chromic polynicotinate, chromium chloride, chromium histidinate, chromium nicotinate-glycinate, chromium phenylalanine or chromium yeast. The method may further comprise administering a cholinesterase inhibitor, memanitine, vitamin E, a phospholipid or an omega-3 fatty acid to the individual.

In one embodiment, a method for supporting cognitive function in an individual in need thereof is provided, the method comprising administering to the individual an effective amount of a dietary chromium complex. In another embodiment, the chromium complex is chromium picolinate, chromic tripicolinate, chromium nicotinate, chromic polynicotinate, chromium chloride, chromium histidinate, chromium nicotinate-glycinate, chromium phenylalanine or chromium yeast. The method may further comprise administering a cholinesterase inhibitor, memanitine, vitamin E, a phospholipid or an omega-3 fatty acid to the individual.

In one embodiment, a method for supporting cognitive function related to brain insulin levels in an individual is provided, the method comprising administering to the individual an amount of a dietary chromium complex effective to increase brain insulin levels. In another embodiment, the chromium complex is chromium picolinate, chromic tripicolinate, chromium nicotinate, chromic polynicotinate, chromium chloride, chromium histidinate, chromium nicotinate-glycinate, chromium phenylalanine or chromium yeast. The method may further comprise administering a cholinesterase inhibitor, memanitine, vitamin E, a phospholipid or an omega-3 fatty acid to the individual.

In one embodiment, a method for supporting cognitive function in an individual is provided, wherein the individual has brain insulin resistance, the method comprising administering an amount of a chromium complex to the individual effective to decrease the brain insulin resistance. In another embodiment, the chromium complex is chromium picolinate, chromic tripicolinate, chromium nicotinate, chromic polynicotinate, chromium chloride, chromium histidinate, chromium nicotinate-glycinate, chromium phenylalanine or chromium yeast. The method may further comprise administering a cholinesterase inhibitor, memanitine, vitamin E, a phospholipid or an omega-3 fatty acid to the individual.

In some embodiments, a method of treating a cognitive condition is provided, the method including: identifying a mammal suffering from a cognitive condition; and administering an effective amount of a chromium complex, or pharmaceutically acceptable salt thereof, to said mammal. The cognitive condition may comprise Alzheimer's disease ormild cognitive impairment and/or may be associated with reduced memory functioning and/or reduced brain insulin levels. The amount may be effective to inhibit cognitive degradation and/or to slow progression of memory impairment. The chromium complex may be selected from the group consisting of chromium picolinate, chromic tripicolinate, chromium nicotinate, chromic polynicotinate, chromium chloride, chromium histidinate, chromium yeast, chromium nicotinate-glycinate, chromium phenylalanine. The method may further include administering a cholinesterase inhibitor, memanitine, vitamin E, phospholipid or omega-3 fatty acid to said individual. The mammal may be a human.

In some embodiments, a method of treating a cognitive condition is provided, the method including identifying a mammal at risk of suffering from a cognitive condition; and administering an effective amount of a chromium complex, or pharmaceutically acceptable salt thereof, to said mammal. The mammal may have a genetic risk factor comprising a mutated gene and/or a family history of the cognitive condition. The amount may be effective to delay onset of the cognitive condition. The chromium complex may be selected from the group consisting of chromium picolinate, chromic tripicolinate, chromium nicotinate, chromic polynicotinate, chromium chloride, chromium histidinate, chromium nicotinate-glycinate, chromium phenylalanine, chromium yeast. The method may further include administering a cholinesterase inhibitor, memanitine, vitamin E, phospholipid or omega-3 fatty acid to said individual.

In some embodiments, a method of treating memory impairment is provided, the method including: identifying a mammal suffering from age-related memory impairment; and administering an effective amount of a chromium complex, or pharmaceutically acceptable salt thereof, to said mammal. The mammal may be at least about 70 years old. The amount may be effective to inhibit progression of long-term memory impairment and/or to inhibit progression of long-term memory impairment. The chromium complex may be selected from the group consisting of chromium picolinate, chromic tripicolinate, chromium nicotinate, chromic polynicotinate, chromium chloride, chromium histidinate, chromium nicotinate-glycinate, chromium phenylalanine, chromium yeast. The method may further include administering a cholinesterase inhibitor, memanitine, vitamin E, phospholipid or omega-3 fatty acid to said individual.

In some embodiments, a method for supporting cognitive function is provided, the method including: providing a dietary chromium complex to an individual; advising the individual that the dietary chromium complex may support cognitive function; and administering or self-administering the dietary chromium complex to the individual. The chromium complex may be selected from the group consisting of chromium picolinate, chromic tripicolinate, chromium nicotinate, chromic polynicotinate, chromium chloride, chromium histidinate, chromium nicotinate-glycinate, chromium phenylalanine, chromium yeast. The method may further include administering a cholinesterase inhibitor, memanitine, vitamin E, phospholipid or omega-3 fatty acid to said individual.

In some embodiments, a use of a dietary chromium complex in the preparation of a medicament for treating impaired cognition is provided. In some embodiments, dietary chromium complex for treating impaired cognition is provided.

Also contemplated are the use of any nutritional or dietary chromium complex, including any of the foregoing, in the preparation of a medicament for treating impaired cognition, including any of the cognitive conditions described herein. Further contemplated is a nutritional or dietary chromium complex for treating a cognitive condition or impaired cognition. Again, it is contemplated to use any of the chromium complexes, including those described herein, for treating each of the various cognitive conditions discussed herein.

### BRIEF DESCRIPTION OF THE DRAWINGS

FIGS. 1A-1D show the average escape latencies in a Morris Swim Maze for rats receiving no treatment or one of three chromium treatments.

FIGS. 2A and 2B show the difference in escape latencies in a Morris Swim Maze for rats receiving one of three chromium treatments as compared to control rats.

FIGS. 3A and 3B show the percent change in escape latencies in a Morris Swim Maze for rats receiving one of three chromium treatments as compared to control rats.

FIGS. 4A and 4B show the percent time that rats spent in a target quadrant during a probe trial of a Morris Swim Maze experiment.

FIG. 5 shows the free recall intrusion score for subjects receiving a placebo or chromium treatment, before and after the treatment.

FIG. 6 shows a motor speed for subjects receiving a placebo or chromium treatment, before and after the treatment.

FIGS. 7A and 7B show fMRI data for subjects receiving a placebo or chromium treatment, before and after the treatment.

FIG. 8 shows fMRI data for a subject receiving a chromium picolinate treatment.

FIG. 9 shows fMRI data for a subject showing activation in left prefrontal cortex during a working memory task.

### DETAILED DESCRIPTION OF THE PREFERRED EMBODIMENTS

Embodiments provide methods of treating or preventing impaired memory and/or cognitive function by identifying an individual in need of such treatment, and administering one or more chromium complexes to the individual. The term “administering” includes administration by another individual (e.g., a physician, caregiver or family member), or self-administration. The chromium complex may be administered as a pharmaceutical formulation or nutritional supplement. The impaired memory and/or cognitive function may be associated with various disorders, including Alzheimer's disease, dementia, amnesia, short term memory loss, mild cognitive impairment (MCI) and loss of cognitive function in individuals with diabetes.

MCI is recognized as an age-associated condition representing the initial clinical manifestation of the neurodegenerative processes associated with Alzheimer's disease. MCI has been conceptualized as a transition stage in the course of progressive decline to dementia. MCI is defined as significant memory impairment in the absence of generalized intellectual and functional deficiency and in the absence of diagnosed dementia. While mild cognitive impairment can affect many areas of cognition, such as language, attention, reasoning, judgment, reading and writing, most research has focused on its effects on memory. The disorder can be divided into two broad subtypes. Amnestic MCI significantly affects memory, while nonamnestic MCI does not. Other functions, such as language and attention span, may be impaired in either subtype.

Amnestic MCI has been linked to Alzheimer's disease, while nonamnestic MCI may progress to other types of syndromes such as frontotemporal dementia, primary progressive aphasia or dementia with Lewy bodies. However, some individuals with MCI do not develop any type of dementia. Some remain stable, while others even revert to normal. Preliminary reports from a recent Mayo Clinic study suggest that about 12 percent of those over the age of 70 have mild cognitive impairment. People with MCI are three to four times more likely to develop Alzheimer's than those without such impairment.

As described in more detail herein, chromium picolinate supplementation has been shown to increase brain insulin, tryptophan and melatonin levels, and to decrease cortisol levels in a rat model of diabetes, which is an art-accepted animal model of human diabetes. In some embodiments, methods are provided for increasing brain insulin levels, tryptophan levels and/or melatonin levels, and/or for decreasing cortisol levels, by administering one or more chromium complexes to an individual in need of increased brain insulin, tryptophan and melatonin levels, and decreased cortisol levels. Insulin resistance in the brain may be a contributing factor in the decline in memory/cognitive function that occurs in Alzheimer's disease. In some embodiments, chromium complexes are provided to treat impaired memory and/or cognitive function. Chromium complexes can also be used prophylactically to at least partially prevent loss of memory and/or cognitive function. Thus use of chromium complexes to treat impaired memory and/or cognitive function associated with any disorder or condition is within the scope of the embodiments described herein.

The chromium complexes disclosed herein may also be used to generally support cognitive function, inhibit or slow cognitive decline, improve memory, inhibit or reduce memory degradation, treat symptoms of Alzheimer's disease, inhibit or slow the development of Alzheimer's disease, and/or treat mild cognitive impairment. Dietary chromium complex may be provided to an individual. The individual may be advise that the dietary chromium complex may support cognitive function, inhibit or slow cognitive decline, improve memory, inhibit or reduce memory degradation, treat symptoms of Alzheimer's disease, inhibit or slow the development of Alzheimer's disease, and/or treat mild cognitive impairment. The dietary chromium complex may be administered to the individual. In one embodiment, the need for supporting cognitive function is related to reduced brain insulin levels in an individual, wherein the chromium complex is effective to increase brain insulin levels. In another embodiment, the need for supporting cognitive function, inhibiting or slowing cognitive decline, improving memory, inhibiting or reducing memory degradation, treating symptoms of Alzheimer's disease, inhibiting or slowing the development of Alzheimer's disease, and/or treating mild cognitive impairment is due to increased brain insulin resistance in an individual, wherein the chromium complex is effective to decrease the brain insulin resistance.

In some embodiments, a chromium complex is administered or provided to a patient.

The patient may be diagnosed with a cognitive-related, insulin-related, and/or memory-related disorder. The patient may, for example, be diagnosed with Alzheimer's disease or mild cognitive impairment.

The patient may have reduced cognitive abilities. The reduced cognitive abilities may comprise reduced memory functioning. The reduced cognitive abilities may be associated with a disease, such as Alzheimer's disease or mild cognitive impairment.

The patient may be given a neuropsychological test, such as a test described in Example 3 or a test listed below. The neuropsychological test may determine whether the patient suffers from a reduced cognitive ability. A chromium complex may be administered to the patient based at least partly on a result of the test (e.g., a below-threshold score).

A chromium complex may be administered to an elderly patient. The patient may be at least about 40, 45, 50, 55, 60, 65, 70, 75, 80, 85, 90, 95, 100, 105 or 110 years old. The complex may slow or inhibit age-related memory and/or cognitive degradation.

The patient may have a family history of a memory- or cognitive-related disease, such as Alzheimer's. The patient may have a genetic risk of a memory- or cognitive-related disease, such as Alzheimer's. For example, the patient may have a mutated gene, such as a mutated APP, PS1 and/or PS2 gene. The patient may have an E4 form of the APOE gene.

In some instances, the patient is healthy, not suffering from a cognitive-related disease, not suffering from a memory-related disease, and/or not elderly.

In some embodiments, the determination of whether the complex will be administered is based on a test result (e.g., a neuropsychological test result, a genetic screening test result or a neurological imaging test result), the patient's age, or a diagnosis. In some instances, the dosage of the complex administered is based on a test result (e.g., a neuropsychological test result, a genetic screening test result or a neurological imaging test result), the patient's age, or a diagnosis. Improvement in memory and/or cognition may determined using standard neuropsychological tests, including, but not limited to, any of the tests described in Example 3, the Bender-Gestalt test, the Benton Visual Retention test, the Clinical Dementia rating, the Continuous Performance task, the Hayling and Brixton tests, the Lexical decision task, the Mini mental state examination, the Stroop task, the Weschler Adult Intelligence Scale or the Wisconsin card sorting task.

In some instances, a chromium complex effectively slows or stabilizes neurological changes (e.g., in atrophy) observed using imaging techniques (e.g., PET, MRI, or fMRI) that are associated with the progression of Alzheimer's disease.

In some instances, a chromium complex improves or inhibits the degradation of long- and/or short-term memory.

It should be noted that by “improvement” of memory and/or cognitive function, it is meant that an individual exhibits at least about a 5%, 10%, 15%, 20%, 25%, 30%, 35%, 40%, 45%, 50%, 55%, 60%, 65%, 70%, 75%, 80%, 85%, 90%, 95%, or 100% improvement in any one or more test(s) used to measure memory and/or cognitive function.

The “slowing” or “inhibiting” of a condition refers to an instance in which an individual receiving a treatment described herein exhibits a change that is less than about 100%, 95%, 90%, 85%, 80%, 75%, 70%, 65%, 60%, 55%, 50%, 45%, 40%, ,35%, 30%, 25%, 20%, 15%, 10% or 5% than would be expected (e.g., based on the individual's medical history or a control group) if the individual was not receiving the treatment. For example, if a person suffering from Alzheimer's disease is expected to experience 10% greater atrophy within a year but instead experiences a 5% greater atrophy, the atrophy may be referred to as being slowed or inhibited.

In one embodiment, a chromium complex is administered to a patient in need of treatment of impaired memory and/or cognitive function in combination with one or more compounds conventionally used to treat this condition, such as cholinesterase inhibitors, memanitine, vitamin E supplementation, phospholipid supplementation, omega-3 fatty acid supplementation. Other phospholipids and omega-3 fatty acids may also be used in combination with a chromium picolinate complex. Other phospholipids include phosphatidyl ethanolamine, phosphatidylglycerol and phosphatidylcholine (lecithin). Other omega-3 fatty acids include α-linolenic acid (ALA). The one or more compounds may be co-administered with the chromium picolinate complex as separate pharmaceutical formulations or nutritional supplements, or may be incorporated into the same pharmaceutical formulation or nutritional supplement (e.g., in the same tablet or capsule as the chromium complex). If the chromium complex and other agent are administered in separate pharmaceutical formulations or supplements, they may be administered at the same time, the chromium complex may be administered before the other agent or vice versa.

Because chromium complexes are also demonstrated herein to increase levels of melatonin, these compounds may also be useful as sleep aids, to prevent jet lag and to treat autism. In addition, their beneficial effect on cortisol levels indicate their utility in reducing stress and stress-related disorders such as post-traumatic stress disorder (PTSD).

The terminology used in the description presented herein is not intended to be interpreted in any limited or restrictive manner, simply because it is being utilized in conjunction with a detailed description of certain specific embodiments described herein. Furthermore, embodiments described herein can include several novel features, no single one of which is solely responsible for its desirable attributes or which is essential to practicing the invention herein described.

The term “chromium complex” refers to a coordination compound in which chromium is covalently or noncovalently bound to an organic or inorganic molecule. Chromium complexes include, but are not limited to, chromium picolinate, chromic tripicolinate, chromium nicotinate, chromic polynicotinate, chromium chloride, chromium histidinate, chromium nicotinate-glycinate, chromium phenylalanine, chromium yeast, or any other chromium complex, whether now known or developed in the future.

The term “patient” refers to an animal which can be treated using the compositions and methods of the invention. Examples of animals include mammals, such as mice, rabbits, rats, horses, goats, dogs, cats, pigs, cattle, sheep, and primates (e.g. chimpanzees, gorillas, and, preferably, humans).

The term “treating” or “treatment” does not necessarily mean total curing. Any alleviation of any undesired signs or symptoms of the disease to any extent or the slowing down of the progress of the disease can be considered treatment.

As used herein, the phrase “over a period of time,” can refer to a period of minutes, hours or days. For example, over a period of time can refer to at least 10 minutes, at least 15 minutes, at least 30 minutes, at least 60 minutes, at least 75 minutes, at least 90 minutes, at least 105 minutes, at least 120 minutes, at least 3 hours, at least 4 hours, at least 5 hours, at least 6 hours, at least 7 hours, at least 8 hours, at least 9 hours, at least 10 hours, at least 12 hours, at least 14 hours, at least 16, hours, at least 18 hours, at least 20 hours, at least 22 hours, at least one day, at least two days, at least three days, at least 4 days, at least 5 days, at least 6 days, at least a week, or any interval of time in between. In other words, the chromium from the composition can be absorbed by the individual to whom it is administered over a period of at least 10 minutes, at least 15 minutes, at least 30 minutes, at least 60 minutes, at least 75 minutes, at least 90 minutes, at least 105 minutes, at least 120 minutes, at least 3 hours, at least 4 hours, at least 5 hours, at least 6 hours, at least 7 hours, at least 8 hours, at least 9 hours, at least 10 hours, at least 12 hours, at least 14 hours, at least 16, hours, at least 18 hours, at least 20 hours, at least 22 hours, at least one day, at least two days, at least three days, at least 4 days, at least 5 days, at least 6 days, at least a week, or any interval of time in between.

In another example, over a period of time can refer to about 10 minutes, about 15 minutes, about 30 minutes, about 60 minutes, about 75 minutes, about 90 minutes, about 105 minutes, about 120 minutes, about 3 hours, about 4 hours, about 5 hours, about 6 hours, about 7 hours, about 8 hours, about 9 hours, about 10 hours, about 12 hours, about 14 hours, about 16, hours, about 18 hours, about 20 hours, about 22 hours, about one day, about two days, about three days, about 4 days, about 5 days, about 6 days, about a week, or any interval of time in between.

As used herein, a composition that “substantially” comprises a compound means that the composition contains more than about 80% by weight, more preferably more than about 90% by weight, even more preferably more than about 95% by weight, and most preferably more than about 97% by weight of the compound. As used herein, a composition that “substantially” comprises a chromium complex refers to a composition that contains more than or equal to 7.0% of trivalent or dietary chromium. Preferably, a certificate of analysis for the compositions disclosed herein indicate that the compositions are negative for microbial growth, yeast and mold should be present in less than 300 cells/g and the toxic metals should be less than 1 ppm.

In some embodiments, the compositions disclosed herein are in the form of pharmaceutically acceptable salts. The phrase “pharmaceutically acceptable salt(s),” is art recognized and, as used herein includes, but is not limited to, salts of acidic or basic groups that may be present in the compositions disclosed herein. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds present in the compositions disclosed herein that include an amino moiety also can form pharmaceutically acceptable salts with various amino acids, in addition to the acids mentioned above. Compounds present in the compositions disclosed herein that are acidic in nature are capable of forming base salts with various pharmacologically acceptable cations. Non-limiting examples of such salts include alkali metal or alkaline earth metal salts and, particularly, calcium, magnesium, sodium lithium, zinc, potassium, silicon, phosphorus and iron salts.

As used herein, the term “hydrate” means a compound or a salt thereof, that further includes a stoichiometric or non-stoichiometric amount of water bound by non-covalent intermolecular forces. The term hydrate includes solvates, which are stoichiometric or non-stoichiometric amounts of a solvent bound by non-covalent intermolecular forces. Preferred solvents are volatile, non-toxic, and/or acceptable for administration to humans in trace amount.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients; such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. In one embodiment, the pharmaceutically acceptable carrier is suitable for intravenous administration. In another embodiment, the pharmaceutically acceptable carrier is suitable for locoregional injection.

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted-into esters via treatment with an alcohol in the presence of a catalyst. Hydroxyls can be converted into esters via treatment with an esterifying agent such as alkanoyl halides. The term also includes lower hydrocarbon groups capable of being solvated under physiological conditions, e.g., alkyl esters, methyl, ethyl and propyl esters. (See, for example, Berge et al., supra.)

The language “pharmaceutical composition” is used interchangeably with “therapeutic agent” and includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “nutritional supplement” refers to a composition for use in supplementing the diet by increasing the total dietary intake. In embodiments disclosed herein, the nutritional supplement may comprise chromium complex, or pharmaceutically acceptable salt or ester thereof, which may be present alone, or in admixture with one or more conventional supplement ingredients, including a vitamin, a mineral, an herb or other botanical, an essential amino acid, an essential fatty acid, or a concentrate, metabolite, constituent, extract, or combination of any of the above.

The chromium complexes can be provided as a tablet, aqueous or oil suspension, dispersible powder or granule, emulsion, hard or soft capsule, syrup, elixir, bar or beverage. Compositions intended for oral use can be prepared according to any method known in the art for the manufacture of pharmaceutically acceptable compositions and such compositions may contain one or more of the following agents: sweeteners, flavoring agents, coloring agents and preservatives. The sweetening and flavoring agents may increase the palatability of the preparation. Tablets containing chromium complexes in admixture with non-toxic pharmaceutically acceptable excipients suitable for tablet manufacture are acceptable. Pharmaceutically acceptable vehicles such as excipients are compatible with the other ingredients of the formulation (as well as non-injurious to the patient). Such excipients include inert diluents such as calcium carbonate, sodium carbonate, lactose, calcium phosphate or sodium phosphate; granulating and disintegrating agents, such as corn starch or alginic acid; binding agents such as starch, gelatin or acacia; and lubricating agents such as magnesium stearate, stearic acid or talc. Tablets can be uncoated or can be coated to, for example, delay disintegration and absorption in the gastrointestinal tract and thereby provide a sustained action over a longer period of time. For example, a time delay material such as glyceryl monostearate or glyceryl distearate alone or with a wax can be employed. The tablet, capsule, gel cap or caplet is manufactured by a standard process, for example, in the case of tablet, direct compression or a wet or dry granulation process.

In some embodiments, the compositions disclosed herein are formulated for oral delivery, for example in the form of tablets, lozenges, aqueous or oily suspensions, granules, powders, emulsions, capsules, syrups, or elixirs. Compounds and compositions described herein for oral delivery can also be formulated in foods and food mixes. Orally administered compositions can contain one or more optional agents, for example, sweetening agents such as fructose, aspartame or saccharin; flavoring agents such as peppermint, oil of wintergreen, or cherry; coloring agents; and preserving agents, to, for example, provide a pharmaceutically palatable preparation. Moreover, when in tablet or pill form, the compositions can be coated to, for example, delay or extend disintegration and/or absorption in the gastrointestinal tract, which may thereby provide a sustained action over an extended period of time. Selectively permeable membranes surrounding an osmotically active driving compound are also suitable for orally administered compounds and compositions described herein. In these later platforms, fluid from the environment surrounding the capsule is imbibed by the driving compound, which swells to displace the agent or agent composition through an aperture. These delivery platforms can provide an essentially zero-order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, etc. Such vehicles are preferably of pharmaceutical grade.

The chromium complexes are generally be administered in admixture with a suitable pharmaceutical excipient, diluent, and/or carrier selected with regard to the intended route of administration and standard pharmaceutical practice. For example, the chromium complex can be administered orally, buccally or sublingually in the form of tablets, capsules (including soft gel capsules), ovules, elixirs, solutions or suspensions, which may contain flavoring or coloring agents, for immediate-, delayed-, modified-, sustained-, or controlled-release delivery applications. Modified-release dosage forms can contain excipients such as those detailed for immediate-release dosage forms together with additional excipients that act as release-rate modifiers, these being coated on and/or included in the body of the device. Release-rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified-release dosage forms may contain one or a combination of release-rate modifying excipients. Release-rate modifying excipients can be present both within the dosage form, i.e. within the matrix, and/or on the dosage form, i.e. upon the surface or coating.

The tablet, capsule, gel cap, or caplet can contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents, such as magnesium stearate, stearic acid, glyceryl behenate and talc, can also be included. The chromium complexes can be mixed with additional components such as binders, surfactants, fillers, disintegrating agents, alkaline additives, or other pharmaceutically acceptable ingredients, alone or in mixtures. The binders are, for example, celluloses such as hydroxypropyl methylcellulose, hydroxypropyl cellulose, and carboxymethyl-cellulose sodium, polyvinyl pyrrolidone, sugars, starches, and other pharmaceutically acceptable substances with cohesive properties. In some embodiments, pharmaceutical constituents such as binders, fillers, lubricants, distintegrating agents, surfactants, and other pharmaceutically acceptable additives are likewise incorporated into the formulation.

Solid compositions of a similar type can also be employed as fillers in gelatin capsules. Preferred excipients in this regard include lactose, starch, a cellulose, milk sugar or high molecular weight polyethylene glycols. For aqueous suspensions and/or elixirs, the compounds of the invention can be combined with various sweetening or flavoring agents, coloring matter or dyes, with emulsifying and/or suspending agents and with diluents such as water, ethanol, propylene glycol and glycerin, and combinations thereof

The chromium complexes may also be administered via fast-dispersing or fast-dissolving dosages forms. Fast-dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, and/or xylitol.

While oral administration is preferred, the chromium picolinate complexes can also be administered parenterally, for example, intracavernosally, intravenously, intra-arterially, intraperitoneally, intrathecally, intraventricularly, intraurethrally intrasternally, intracranially, intramuscularly or subcutaneously, or it may be administered by infusion techniques. For such parenteral administration, the dosage components are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art

In another embodiment, the oral dosage form may further include an outer coating. The outer coating serves to provide an immediate-release, controlled-release, delayed-release, or enteric-coating to control or delay the release of the chromium complex. The outer coating layer (or layers) can be applied by coating or layering procedures which are well-established in the relevant arts. The outer coating layer is at least one of a pharmaceutically acceptable compound selected from the group consisting of sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium and combinations thereof. Additives including plasticizers, colorants, pigments, fillers, anti-tacking and anti-static agents can optionally be included in the outer coating.

In certain embodiments, the outer coating regulates release of the chromium complex from the tablet, capsule, gel cap, or caplet. Controlled-release, delayed-release, and/or enteric-coating technology is well-established in the pharmaceutical and formulation arts. It may be advantageous to both the patient and the physician that medication be formulated so that it may be administered in a minimum number of daily doses from which the drug is uniformly released over a desired extended period of time. This effect is accomplished using sustained- or slow-release compositions. Sustained- or slow-release compositions containing pharmaceutical medicaments or other active ingredients are designed to contain higher concentrations of the medicament and are prepared in such a manner as to affect sustained or slow release into the gastrointestinal digestive tract of humans or animals over an extended period of time.

Well absorbed oral sustained- or slow-release therapeutic drug dosage forms have inherent advantages over conventional, immediate release dosage forms. The advantages include less frequent dosing of a medicament and resultant patient regime compliance, a more sustained drug blood level response, therapeutic action with less ingested drug and the mitigation of side effects. By providing a slow and steady release of the medicament over time, absorbed drug concentration spikes are mitigated or eliminated by affecting a smoother and more sustained blood level response.

Various hydrophilic and hydrophobic materials, including polymers, can be utilized in preparing sustained release formulations. These formulations are prepared by various methods well-established in the tableting arts, such as solvent evaporation, heat melting, direct compression and wet granulation. In some embodiments, waxes and lipids are used as coating material to retard the release of drugs. The common methods of manufacturing sustained-release medicaments in oral dosage forms using waxes as the controlled-release material admixed with the medicament are (a) melting the drug and wax together, then cooling and milling the melt, and finally tableting after mixing with excipient; (b) using wet granulation techniques, employing an organic solvent as a granulating medium; (c) mixing the drug and waxes in a high shear mixture and using the heat produced during the processing to achieve a homogenous mixture; and (d) using heat radiation to effect melting of the wax in the presence of the drug.

In one embodiment of the invention, controlled-release coatings are prepared by forming a matrix by entrapping the chromium complex in excipients. Diffusion and/or erosion operate to release the chromium complex depending on the properties of the chromium complex and the polymer incorporated in the formulation. One particular attempt at controlled release is detailed in European Patent publication 0 593 309 A2 to Columbo, hereby incorporated by reference in its entirety. The publication shows a three-layer system consisting of two external swelling layers separated by an interposed soluble layer, and a two-layer system consisting of a swellable layer adjacent a soluble and/or erodible layer. The swellable layer(s) consist of methyl cellulose, carboxymethylcellulose sodium, crosslinked carboxymethylcellulose sodium, crosslinked hydroxypropylcellulose, hydroxypropylmethylcellulose, carboxymethyl starch, polymethacrylate, polyvinylpyrrolidone, polyvinyl alcohols, polyethylene glycols, or potassium methacrylate-divinyl benzene copolymer and mixtures thereof

In one embodiment, the soluble and/or erodible layer includes hydroxyethylcellulose, carboxymethylcellulose, alginates, albumin, soluble starch and/or gelatin, mixed with at least one soluble excipient such as saccharide and polyalcohol. In one embodiment, the swellable layer(s) contain an active therapeutic agent. As the swellable layers swell and the erodible layer erodes, the therapeutic agent is released from the swellable layers. In another embodiment, the swellable layer(s) contain a nutritional supplement. In yet another embodiment, the swellable layer(s) contain both the active therapeutic agent and the nutritional supplement.

In certain embodiments, a unit dosage form includes an enteric coating layer. The enteric coating layer(s) are applied using a suitable coating technique. The enteric coating layer material can be dispersed or dissolved in either water or in suitable organic solvents. As enteric coating layer polymers, one or more, separately or in combination, of the following can be used; e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). The enteric coating layers contain pharmaceutically acceptable plasticizers to obtain the desired mechanical properties such as flexibility and hardness of the enteric coating layers. Such plasticizers are, for instance, but not restricted to, triacetin, citric acid esters, phthalic acid esters, dibutyl sebacate, cetyl alcohol, polyethylene glycols, polysorbates or other plasticizers. The amount of plasticizer is optimized for each enteric coating layer formula in relation to selected enteric coating layer polymer(s), selected plasticizer(s) and the applied amount of the polymer(s), in such a way that the mechanical properties such as flexibility and hardness of the enteric coating layers, are adjusted so that the acid resistance of the formulation does not decrease significantly during the compression of the components into tablets, for example. The amount of plasticizer is usually above 10% by weight of the enteric coating layer polymer(s), preferably 15-50%, and more preferably 20-50%. Additives such as dispersants, colorants, pigments, polymers, anti-tacking and anti-foaming agents can likewise be included into the enteric coating layer(s). In some embodiments, other compounds can be added to increase film thickness and to decrease diffusion.

The present invention further includes a chromium complex formulated in a capsule. Capsule formulation is well-established in the pharmacological arts. See, e.g., U.S. Pat. No. 3,965,256 to Leslie, the entire contents of which are hereby incorporated by reference. Slow release capsules are prepared by filling the appropriate quantity of the above-described tablet granulation mixture into gelatin capsules of suitable size and shape, with slight modification such as, for example, eliminating the tablet lubricant or the tablet binder. A slow-release capsule may contain a mixture of the appropriate quantity of the combination of higher aliphatic alcohol and hydrated hydroxy-alkyl cellulose together with the active ingredients (a therapeutic agent and a nutritional supplement) and diluent. The diluent serves to achieve the appropriate concentration of the slow-release composition within the unit dosage form. As for the slow-release tablet preparations, the time span for the release of the active ingredient in the capsule formulation will depend upon the concentration of the slow-release composition within the total weight of the capsule formulation.

Formulations for oral use can be presented as hard gelatin capsules wherein the active ingredient(s) are mixed with an inert solid diluent, for example calcium carbonate, calcium phosphate or kaolin, or as soft gelatin capsules wherein the active ingredient is mixed with water or an oil medium, such as peanut oil, liquid paraffin or olive oil. Aqueous suspensions can contain the chromium complex of the invention in admixture with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin.

In another embodiment, the chromium complex is formulated as a softgel capsule. Oil suspensions can be formulated by suspending the active ingredient in a vegetable oil, such as arachis oil, olive oil, sesame oil or coconut oil, or in a mineral oil such as liquid paraffin. The oil suspension can contain a thickening agent, such as beeswax, hard paraffin or cetyl alcohol. Sweetening agents, such as those set forth above, and flavoring agents can be added to provide a palatable oral preparation. These compositions can be preserved by an added antioxidant such as ascorbic acid. Dispersible powders and granules of the invention suitable for preparation of an aqueous suspension by the addition of water provide the active ingredient in admixture with a dispersing or wetting agent, a suspending agent, and one or more preservatives. Additional excipients, for example sweetening, flavoring and coloring agents, can also be present.

Syrups and elixirs can be formulated with sweetening agents, such as glycerol, sorbitol or sucrose. Such formulations can also contain a demulcent, a preservative, a flavoring or a coloring agent.

It will be appreciated by the skilled artisan that the amount of therapeutic agent in combination with nutritional supplement that can be combined with the carrier material to produce a single dosage form will vary depending upon the host treated and the particular mode of administration.

When administered to a mammal, e.g., to an animal for veterinary use or for improvement of livestock, or to a human for therapeutic use, the compositions disclosed herein are administered in isolated form or as the isolated form in a therapeutic composition. As used herein, “isolated” means that the compositions disclosed herein are separated from other components of either (a) a natural source, such as a plant or cell or food, preferably bacterial culture, or (b) a synthetic organic chemical reaction mixture. Preferably, via conventional techniques, the compositions disclosed herein are purified. As used herein, “purified” means that when isolated, the isolate contains at least 95%, preferably at least 98% of the composition.

The amount of a compound of the invention that will be effective in the treatment of a particular disorder or condition disclosed herein will depend on the nature of the disorder or condition, and can be determined by standard clinical techniques. As used herein, the term “treatment” or “treating” refers to an amelioration of a disease or disorder, or at least one discernible symptom thereof. The term “treatment” or “treating” refers to inhibiting the progression of a disease or disorder, either physically, e.g., stabilization of a discernible symptom, or physiologically, e.g., stabilization of a physical parameter, or both.

In addition, in vitro or in vivo assays may optionally be employed to help identify optimal dosage ranges. The precise dose to be employed in the compositions may also depend on the route of administration, and the seriousness of the disease or disorder, and should be decided according to the judgment of the practitioner and each circumstance. Suitable dosage ranges of metformin are well-established. Suitable dosage ranges for oral administration are generally about 0.001 milligram to 5000 milligrams of a total chromium complex per kilogram body weight. In preferred embodiments, the oral dose is 0.01 milligram total chromium complex to 1000 milligrams per kilogram body weight, more preferably 0.1 milligram to 100 milligrams per kilogram body weight, more preferably 0.5 milligram to 25 milligrams per kilogram body weight, and yet more preferably 1 milligram to 10 milligrams per kilogram body weight. The dosage amounts described herein refer to total amounts administered; that is, if more than one chromium complex or more than one composition disclosed herein is administered, the preferred dosages correspond to the total amount of the compositions disclosed herein administered. Oral compositions preferably contain 10% to 95% active ingredient.

In accordance with the methods disclosed herein, the amount of chromium provided by the compositions that comprise chromium complexes disclosed herein can be between about 25 μg per day and about 10,000m per day In accordance with the methods disclosed herein, the amount of chromium provided by the compositions that comprise chromium complexes disclosed herein can be for example at least 25 μg per day, at least 60 μg, at least 70 μg, at least 80 μg, at least 90 μg, at least 100 μg, at least 125 μg, at least 150 μg, at least 200 μg, at least 250 μg, at least 300 μg, at least 350 μg, at least 400 μg, at least 450 μg, at least 500 μg, at least 550 μg, at least 600 μg, at least 650 μg, at least 700 μg, at least 750 μg, at least 800 μg, at least 850 μg, at least 900 μg, at least 950 μg, at least 1,000 μg, at least 1500 μg, at least 2,000 μg, at least 2500 μg, at least 3000 μg, at least 3500 μg, at least 4000 μg, at least 4500 μg, at least 5,000 μg, at least 5500 μg, at least 6000 μg, at least 6500 μg, at least 7000 μg, at least 7500 μg, at least 8000 μg, at least 8500 μg, at least 900 μg, at least 9500 μg, or at least 10,000 μg chromium complex/day. As discussed above, chromium complexes may be trivalent complexes, such as chromium picolinate, chromic tripicolinate, chromium nicotinate, chromic polynicotinate, chromium chloride, chromium histidinate, chromium nicotinate-glycinate, chromium phenylalanine, chromium yeast, or any other chromium complex, whether now known or to be developed in the future.

By way of example, the level of chromium used for supplementation in order to inhibit the onset of insulin resistance is at least about 25 μg/day. Chromium picolinate and chromium chloride have been administered to rats at levels several thousand times the upper limit of the estimated safe and adequate daily dietary intake (ESADDI) for chromium for humans (based on body weight) without toxic effects. R. Anderson et al., Lack of Toxicity of Chromium Chloride and Picolinate, 16 J. Am. Coll. Nutr. 273-279 (1997). While the level of chromium used for supplementation may be within several thousand times the upper limit of the ESADDI, preferably, the amount of chromium is between about 50 and 2,000 μg/day. More preferably, the amount of chromium is between about 300 and 1,000 μg/day. Most preferably, the amount of chromium is between about 400 and 1,000 μg/day. In a particularly preferred embodiment, the amount of chromium is between about 600 and 1,000 μg/day. These doses are based on a 70 kg adult human, and that the dose can be applied on a per-kilogram basis to humans or animals of different weights.

### Therapeutic/Prophylactic Administration and Compositions

Due to the activity of the compounds and compositions described herein, they are useful in veterinary and human medicine. As described above, the compounds and compositions described herein are useful for the treatment or prevention of impaired memory and cognition, such as the impaired memory and cognition that occurs in Alzheimer's disease, dementia and other disorders.

### Examples 1

### Modulation of Serotonergic Properties and Carbohydrate Metabolism in a Rat Model of Diabetes

An animal model of type 2 diabetes was produced by feeding male Sprague-Dawley rats (n=33) with a high-fat diet (HFD, 40% Kcal from fat) for 2 weeks, then intraperitoneally administering streptozotocin (STZ, 40 mg/kg). The HFD/STZ-treated rats were then administered 80 mcg chromium picolinate/kg body weight/day for 10 weeks. Chromium picolinate was obtained from Nutrition 21, Purchase, N.Y. Untreated Sprague-Dawley (standard control diet) and HFD/STZ-treated rats not administered chromium picolinate served as controls.

HFD/STZ treatment reduced brain chromium levels and impaired all measurements of carbohydrate metabolism and serotonergic properties (Table 1). Chromium picolinate administration to HFD/STZ treated rats increased brain chromium levels and improved all measurements or carbohydrate metabolism and serotonergic properties. Compared to the HFD/STZ group, chromium picolinate significantly increased insulin sensitivity (CISI) and reduced blood glucose and insulin resistance. Chromium picolinate significantly increased brain and serum insulin levels, serum tryptophan, brain and serum serotonin, and melatonin, and decreased cortisol levels. Chromium picolinate administration was well tolerated without any adverse events.

TABLE 1
Effect of different treatments on chromium, carbohydrate
metabolism and serotonergic properties (mean ± SD)

| End Markers | Control^{a} | HFD/STZ^{b} | HFD/STZ + CrPic^{c} |
|---|---|---|---|
| Chromium - | 17.6 ± 1.3 | 5.2 ± 1.4 | 22.6 ± 1.2 |
| Brain (ng/g) | | | |
| CISI | 2.7 ± 0.03 | 0.8 ± 0.04 | 1.3 ± 0.04 |
| Glucose (mg/dL) | 103.0 ± 2.2 | 469.0 ± 7.9 | 287.0 ± 2.7 |
| Insulin | | | |
| Brain (ng/g) | 27.4 ± 5.2 | 3.8 ± 1.2 | 8.9 ± 2.0 |
| Serum (uU/mL) | 48.6 ± 1.2 | 23.2 ± 0.4 | 25.9 ± 0.5 |
| Tryptophan | | | |
| Brain (mcg/g) | 7.5 ± 0.2 | 5.2 ± 1.0 | 5.5 ± 0.2 |
| Serum (mg/dL) | 22.1 ± 0.2 | 12.3 ± 0.3 | 16.1 ± 0.3 |
| Serotonin | | | |
| Brain (mcg/g) | 700.2 ± 12 | 450.3 ± 5.1 | 520.3 ± 4.1 |
| Serum (mg/dL) | 221.9 ± 6.2 | 165.8 ± 1.8 | 174.9 ± 2.5 |
| Melatonin (mcg/g) | 32.0 ± 0.8 | 12.5 ± 0.2 | 16.3 ± 0.2 |
| Cortisol (mg/dL) | 8.2 ± 0.2 | 13.4 ± 0.4 | 11.5 ± 0.4 |

superscripts without a common letter differ; p < 0.01

HFD/STZ treatment lowered brain chromium levels, and impaired glucose metabolism, insulin levels and serotonergic properties. Chromium picolinate administration was effective in restoring these conditions. These results provide a link between defects in glucose metabolism and serotonergic activity. Because insulin resistance in the brain may contribute to a decline in memory/cognition, such as that which occurs in Alzheimer's disease, administration of chromium complexes represents a treatment for impairment of memory and decreased cognitive function.

### Example 2

### Improved Spatial Memory with Chromium Supplementation

8-week old, male, Wistar rats were divided into eight groups: four control groups (C) and four insulin-resistant groups (HFD). Insulin resistance was induced in by feeding the rats a high-fat diet (40% of calories as fat). The four groups in the control and insulin-resistant groups included: a no treatment group, a 14.3 mcg chromium picolinate group (CrPic), a 19.7 mcg chromium histidinate group (CrHis), and a 7.15 chromium picolinate and 9.85 chromium histidinate group (CrPic+CrHis). Four rats were assigned to each of the eight groups. Treatments were administered for twelve weeks.

Spatial learning and memory were then tested using the Morris Water Maze test. Rats were placed in a circular water tank (120 cm diameter, 50 cm height) that was partially filled with water (25° C.). Milk powder was used to render the water opaque. Each rat was acclimated to the task with two days of free-swimming in the pool with no platform. The pool was divided into four quadrants, one of which had a platform submerged about 1.5 cm below the water surface after the accilimation period. Distal spatial cues were provided and kept constant throughout the testing, such that the rats could use the cues to find the platform. Rats were given 60 seconds to find the platform during each trial. The escape latency was defined as the time required for the rat to find a hidden platform. If the rat did not find the platform, the escape latency was defined as the length of the trial.

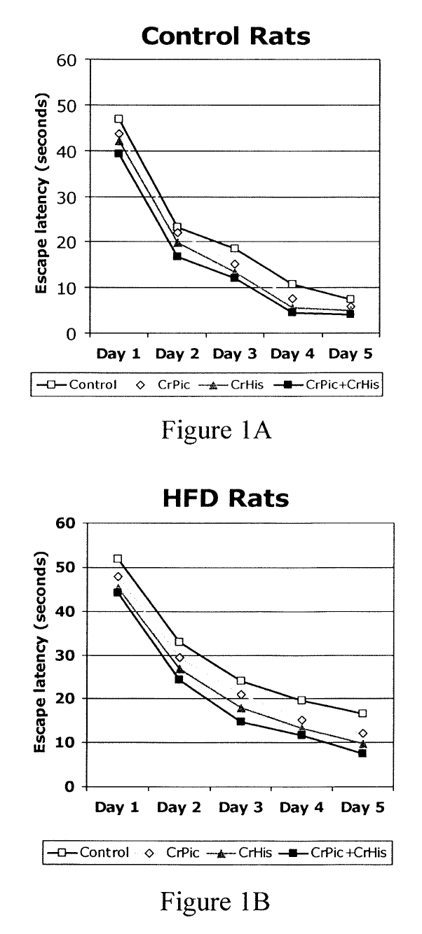

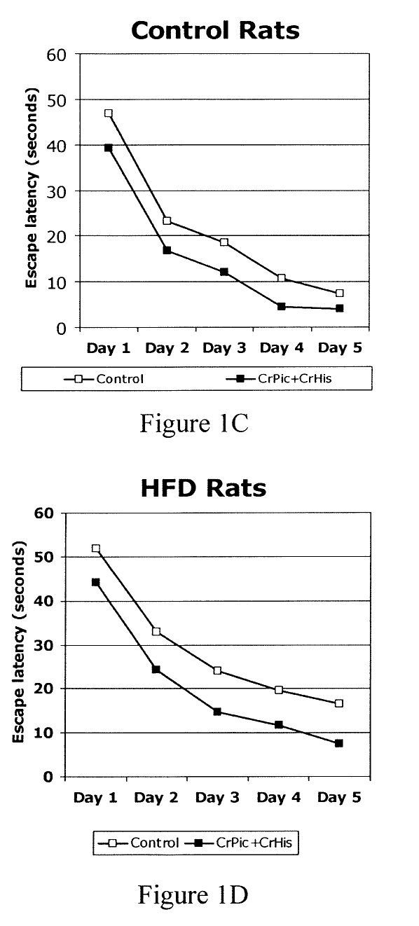

FIGS. 1A-D show the average escape latency calculated across the rats in each group on each day of treatment. In the control rats, the CrPic+CrHis group had significantly shorter escape latencies on days 2-5 as compared to controls, and the CrPic and CrHis groups had significantly shorter escape latencies on day 3 compared to controls. See FIGS. 1A and 1C. In the insulin-resistant rats, the CrPic+CrHis group had significantly shorter escape latencies on days 3-5 as compared to controls, and the CrHis group had significantly shorter escape latencies on days 3 and 5 compared to controls. See FIGS. 1B and 1D.

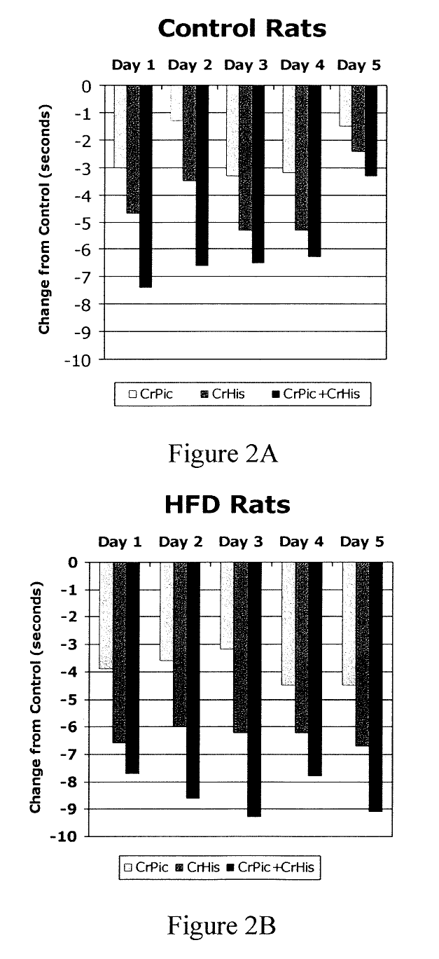

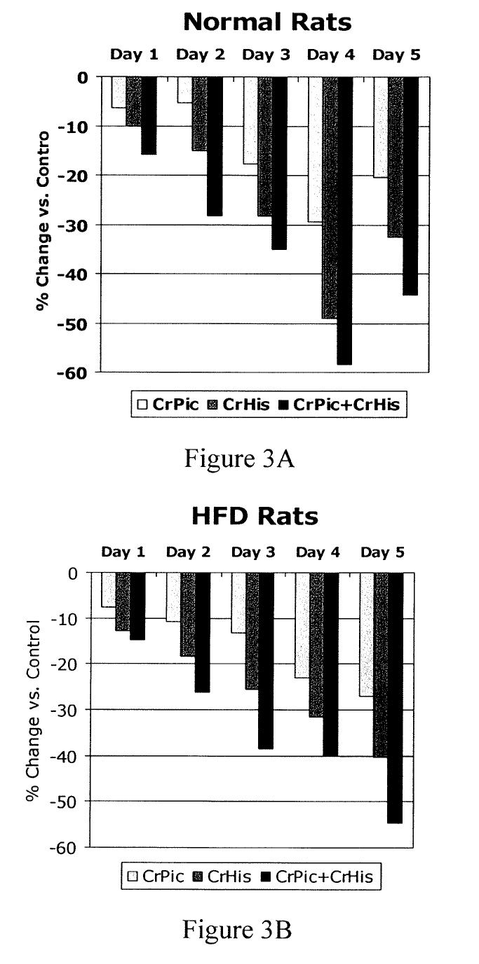

FIGS. 2A and 2B show the difference in the latencies for the CrPic, CrHis and CrPic+CrHis compared to the controls. FIGS. 3A and 3B show the differences in latencies for the CrPic, CrHis and CrPic+CrHis compared to the controls as a percentage change. As shown in FIG. 3B, the treated groups showed larger reduced changes relative to controls during later treatment days. The CrPic+CrHis showed the largest percent reduction among the three groups.

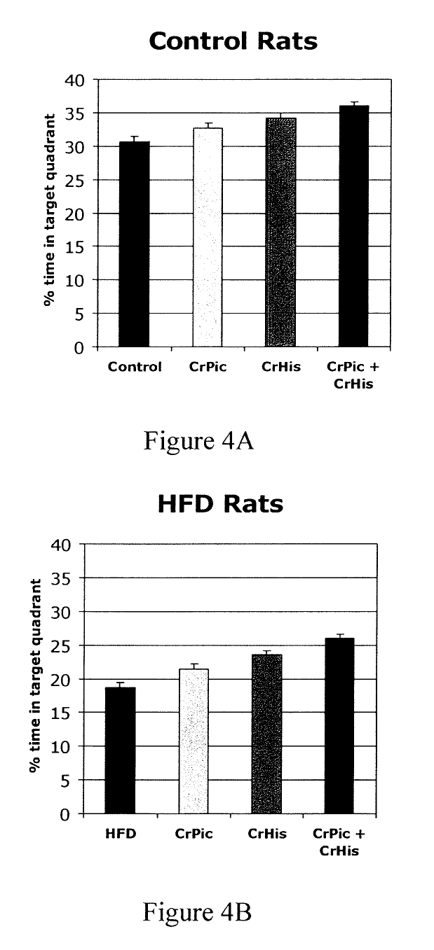

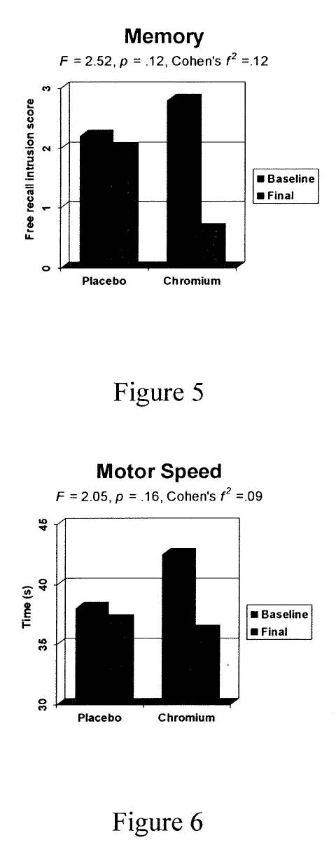

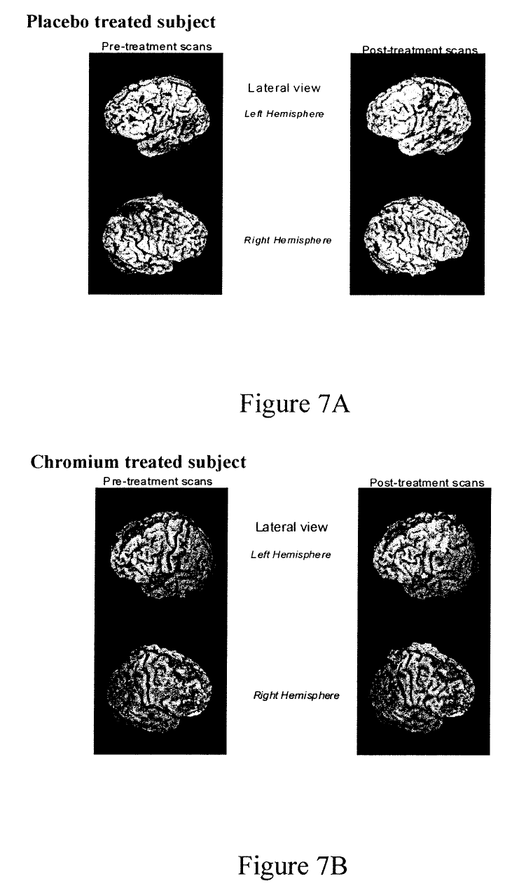

A probe trial was performed in which the hidden platform was removed from the pool, and the animal was allowed to swim for 60 seconds. Typically, a rat will swim in a quadrant of the pool where the platform was previously hidden. FIGS. 4A and 4B show the percentage of time spent in the target quadrant for the eight groups. Rats administered the three treatments spent longer in the target quadrant than control rats.

These findings suggest that chromium histidinate, chromium picolinate and the combination treatment are effective in improving spatial learning and/or memory in control animals and insulin-resistant animals.

### Example 3

### Improvement of Memory and Cognitive Function in an Individual with Alzheimer's Disease

An individual diagnosed with Alzheimer's disease is administered a chromium tripicolinate capsules in an amount that delivers 500 μg chromium per day. The individual takes this dosage once daily for 90 days. During the treatment period, memory and cognition are assessed using one or more of the standard memory tests at 30 days, 60 days and 90 days after beginning treatment. If significant improvement is noted (e.g., 20% or more improvement based on one or more standard neuropsychological memory tests), then this dosage is continued. If no significant improvement is noted (e.g., less than 20% improvement based on one or more standard neuropsychological memory tests), then the dosage of chromium tripicolinate is increased to, for example, 1,000 μg chromium per day and treatment is continued for another 90 days during which memory is assessed at days 30, 60 and 90 of the additional treatment period.

Other methods will be known to the skilled artisan and are within the scope described herein, including processes for preparing the formulations described above.

### Example 4

### Improvement or Memory and Cognitive Function in Age-Related Memory Decline

A randomized, double blind, placebo-controlled trial is conducted to evaluate the efficacy of chromium picolinate in producing enhancements of neuropsychological function in men and women with Mild Cognitive Impairment. Changes in indices of glucose metabolism and in salivary cortisol levels also are measured to gather exploratory information concerning potential mechanisms by which the intervention produces its effects. The subjects participate in a 12-week protocol with major assessments of neuropsychological and biological measures at pre-treatment baseline and during the final week of the intervention. In addition, a brief interim evaluation of neurocognitive function is conducted at 6 weeks to gather information that to assess trends and acute response. Mood, as a potential covariate of the neurobehavioral outcome measures, is also assessed. Overall diet and consumption of other supplements are assessed with diet diaries at different phases of the intervention. Compliance and adverse responses are monitored throughout the period of the intervention by means of weekly telephone contacts.

### Subjects and Plans for Recruitment

The inclusion criteria for subjects includes the following: 1. 65 years of age and older; 2. subjective complaints of mild to moderate forgetfulness and memory retrieval difficulties confirmed by formal assessment; 3. sufficient intelligence and motivation to comprehend and comply with the study protocol. The exclusion criteria are as follows: 1. established dementia or neurological disorder, in particular diagnoses of Alzheimer's Disease, Parkinson's Disease, multi-infarct dementia, or leukoencephalopathy; 2. current or past severe psychiatric disorder such as psychosis or mood disorder requiring hospitalization and/or causing a persisting alteration in level of occupational or social functioning; 3. current treatment with medications or supplements that may affect outcome measures such as insulin sensitizing agents, insulin, antidepressant medications, benzodiazepines, and neuroleptic drugs. All subjects reside in autonomous living situations, including private homes, apartments, and limited assistance retirement communities. Subjects are not recruited from high-support assisted living or nursing home residential settings and subjects who would require this level of residential support, which reflects functional impairment consistent with dementia, are not accepted.

### Procedure

Interested individuals are contacted for screening. During this contact, prospective subjects are provided with an overview of the expectations, timeframe, and compensation for participation in the research. Given positive indications as to the individual's interest, motivation, and ability to participate in the study, two screening instruments are administered.

The first is the Demographic and Medical History Structured Interview Form. This instrument, which requires 15 minutes to complete, inquires about demographic information (including ethnicity and race in accordance with the Office of Management and Budget standards for data collection), inclusion/exclusion criteria, educational, medical, neurological, and psychiatric history, and medication and supplement usage.

The second instrument is the Clinical Dementia Rating Scale (CDR; Hughes et al. 1982), which yields a dementia staging score that is based primarily on memory as well as five other aspects of everyday functioning. It has proven to be useful as an initial screening instrument for dementia and has demonstrated value as a means of predicting progression of cognitive decline. Individuals with CDR scores indicating no memory decline as well as those with moderate or severe impairment are not be invited to participate. Those with scores indicating questionable and mild impairment are included, so that we will select an at-risk sample with early cognitive decline marked primarily by forgetfulness and problems with memory for recent events. In order to verify the prospective subject's report regarding memory functioning, an informant version of the CDR is completed with information provided by an individual who is knowledgeable about the everyday functioning of the prospective subject. Both the participant and informant CDR are reviewed and scored to obtain consensus classification as to dementia stage. This method is recommended as a means of obtaining more objective information about level of functioning and allows establishment of the diagnosis of Mild Cognitive Impairment according to the most well researched criteria for this condition. Furthermore, a more comprehensive staging instrument (the Dementia Rating Scale described below) is administered during the baseline assessment to objectively quantify level of dementia and verify the categorization derived from the CDR.

After reviewing the information gathered in the interviews, the prospective subject is contacted again and the baseline visit will be scheduled. A packet containing a confirmation letter with the date and time of the baseline visit, the container and written instructions for the 7-day diet diary and saliva samples are shipped to each prospective subject. All instructions for at-home activities are reviewed over the telephone when the baseline visit is scheduled. In addition, each individual is contacted by telephone two days before the baseline visit to confirm the appointment and review the instructions for collecting the urine.

Enrollment/baseline visit: During the baseline visit, the informed consent document is completed and subject number assigned. For those individuals who choose not to enroll at that time, the diet diary and saliva samples are destroyed. Otherwise, these items are collected from all enrolled subjects. In addition, the formal dementia rating, mood assessment, and neuropsychological studies are carried out (see instrument descriptions below), and blood samples obtained. A six-week supply of capsules and the 7-day diet diary are distributed. The latter is completed during the week before the interim visit. Finally, the interim visit during the 6th week is scheduled.

Because our subjects will have problems with day-to-day forgetfulness, establishing a practical schedule for capsule ingestion is important. This schedule is developed with each subject individually during the baseline visit and is provided in written form. It indicates time of day that the pills will be taken (typically with the morning meal) and where the supply of tablets will be stored at home. Each subject is contacted by telephone one or two days before each subsequent appointment. Also, a consistent time is determined when the research assistant will contact each subject on a weekly basis throughout the intervention in order to check on compliance and help solve problems that may arise. We have found that these weekly telephone contacts help maintain subject interest in the research and commitment to the regimen.

For subjects that have difficulty with transportation to and from the medical center, the assistance of a spouse, adult child, or close friend is engaged for transportation or some other means of transportation is arranged. For those cases in which transportation to the medical center is not feasible, home evaluation is arranged. In addition, the interim (6^{th} week) visit is performed at home for some of the subjects, if necessary.

Weekly telephone contacts: As noted, the weekly telephone contacts are designed to enhance compliance with the regimen by reviewing capsule ingestion. Patients are asked about any adverse effects and difficulty maintaining the regimen. Also, these contacts are used to confirm subsequent study visits to the medical center.

Interim visit: The subjects return to the medical center during week 6 for pill counts, to obtain the next 6 weeks' supply of capsules, and for a brief cognitive assessment. The cognitive protocol administered during the interim visit includes the CogState assessment and the Verbal Paired Associate Learning test (see below for descriptions of these tasks). The subjects also submit the 7-day diet diary completed for the preceding week and receive diet diary forms to be completed during the week before the final visit.

Final assessment: During the final week of the intervention (week 12 for each subject), the subjects return to the medical center for the termination assessment. This involves repeating the neuropsychological evaluation with alternate forms where indicated below; re-administration of the mood scale; collecting saliva samples, the final 7-day diet diary, and pill containers; and obtaining blood samples. The subjects are debriefed regarding their experiences while participating in the study.

### Major Instruments and Laboratory Tests

| | | | Interim | Final |
|---|---|---|---|---|
| | Pre- | Baseline | Evaluation | Evaluation |
| | Enrollment | Evaluation | (6 weeks) | (12 weeks) |
| Telephone screening | X | | | |
| (inclusion/exclusion | | | | |
| criteria, Clinical | | | | |
| Dementia Rating, | | | | |
| Academic/Medical | | | | |
| History) | | | | |
| 7-day diet diary | | X | X | X |
| Blood samples | | X | | X |
| Saliva samples | | X | | X |
| Mattis Dementia | | X | | |
| Rating Scale | | | | |
| Peabody Picture | | X | | |
| Vocabulary Test | | | | |
| CogState | | X | X | X |
| Stop Signal Task | | X | | X |
| Geriatric Depression | | X | | X |
| Scale | | | | |
| California Verbal | | X | | X |
| Learning Test | | | | |
| Verbal Paired | | X | X | X |
| Associate Learning | | | | |
| 6-week supply of | | X | X | |
| capsules | | | | |
| Subject payment | | X | X | X |

Baseline and final visits include formal assessment of mood and the neuropsychological examination. Because of the repeated psychological assessments, the effect of prior exposure to the procedures on subsequent administrations is an important methodological issue. Practice effect can be understood in terms of both a) enhanced performance because of prior experience with the testing protocols, often termed a procedural practice effect and b) familiarity with the specific item content of a test producing enhanced performance on subsequent administrations. The mood measures generally are not susceptible to these kinds of practice effects because subjects are asked to rate recent subjective experiences. However, the neuropsychological studies of memory function are. Two forms of control are utilized to mitigate practice effects. For those procedures where familiarity with specific content may improve subsequent performance, alternative forms of the tests are used at the different evaluation points. The control for possible procedural practice effects is the comparison of the performance of the active supplement group with that of the placebo group.

Dementia Rating Scale: The Dementia Rating Scale (DRS) is used to more formally evaluate and stage the degree of overall cognitive impairment. It is administered during the baseline visit only. The DRS provides corroborating data regarding the estimate of level of impairment generated from the Clinical Dementia Rating in the screening interview and is a means of characterizing overall level of functioning with an instrument that has demonstrated validity for discriminating and staging level of dementia and for longitudinal assessment. A recent factor analytic study reaffirmed the validity of the DRS factor structure. In cases in which the DRS evaluation disconfirms the Clinical Dementia Rating telephone interview assessment, the DRS score will take precedence.

In order to identify cutoff scores on the DRS suitable for the projected demographic parameters of our sample, several standardization studies of older adults were reviewed, including studies using samples stratified by age, education, and SES and those including African-American samples. It is of interest to note that age is the predominant factor predictive of DRS performance, while gender, race, education, and even cerebrovascular risks were not predictive. Based on these data, we have established a cutoff score of 125 (of a maximum score of 144). Individuals scoring below this level are considered too severely impaired to be included.

Geriatric Depression Scale: The Geriatric Depression Scale is a 30-item inventory designed to assess mood in elderly adults. It is examiner-administered in about 8 minutes to insure accurate rating, although it also can be used in a self-administered format. Prior psychometric studies have resulted in elimination of somatic items that may be related to general ill health and functional limitations in the elderly. The GDS has the advantage of being a largely unitary measure of mood functioning, as factor analysis has indicated one major factor, dysphoria, and minor factors termed worry and apathy. It has been used in a large number of studies of mood and cognition in clinical and nonclinical elderly samples, in particular in age ranges of 55 and older. Consistency and validity data have been collected on several hundred subjects across studies and are satisfactory. It also has been used successfully to discriminate depression among elderly patients with and without early cognitive decline, and normative data are available. The GDS is administered during the baseline and termination assessments primarily as a measure to be used as covariate control for the cognitive outcomes. In addition, it provides direct information as to the effect of the intervention on mood.

Neuropsychological evaluation: The neuropsychological measures described below were selected for two reasons. First, age-related cognitive decline, whether idiopathic or representative of progressive brain-based disease, involves deterioration of executive abilities and episodic memory function. Second, impairment of these cognitive functions is associated with dysfunction in cortical association areas (in particular prefrontal regions) and of medial temporal lobe structures, cerebral structures which are particularly vulnerable to age-related cerebral deterioration and progressive neurodegeneration.

The neuropsychological studies involve measures of executive abilities (working memory capacity and behavioral inhibitory control) and secondary memory function (learning efficiency and retention). The construct, working memory, has guided theory about executive function and generated a great deal of research. Among a number of working memory constructs, inhibitory control and working memory capacity are fundamental. These constructs apply particularly to the study of age-related cognitive decline, whether of so-called normal cognitive aging effects or of progressive dementia and have been related to activation of prefrontal and other cortical regions. Executive ability is evaluated by including instruments that assess working memory capacity and inhibitory control.

In addition, secondary memory function is evaluated with tasks that assess rate of acquisition and retention of new information and associative learning. Memory consolidation ability is fundamental to long-term retention and presumed to be mediated primarily by hippocampal structures of the medial temporal lobe. Diminished rates of acquisition and rapid forgetting are characteristic of age-related memory impairment and of both nonpathological and progressive cognitive decline. Indeed, impairment on measures of ability to learn and remember new information is the single most effective means of detecting cognitive impairment in the elderly. Associative learning is an important memory function that has been examined with respect to normal aging and especially with respect to dementing processes. Impaired ability to form new associations on repeated learning trials has been a cardinal neuropsychological feature of dementia.

The neuropsycholgical measures are administered to each subject during the baseline assessment and again at termination of the study protocol. In addition, an abbreviated neuropsychological assessment is performed at the interim visit during the 6^{th} week to acquire information that may be useful to assess more acute response to the intervention and analyze trends. As described below, equivalent alternate forms of the memory measures will be used at the two evaluation points because these instruments are susceptible to practice effects.

Cog (CogState Ltd, Carlton, Australia): This is set of computer tasks specifically designed to measure working memory, attention, and decision-making in the context of repeated assessment research designs. It is intended for clinical trial use to quantify changes in cognitive ability, even in relatively brief duration studies. It can be repeated as often as required and takes a relatively short time to administer.

The tasks involve a computer display of simple playing cards as stimuli. The stimuli can be randomly assigned in each task and the subject responds by means of one or two keystrokes on the computer keyboard. It is possible to generate an unlimited number of equivalent, alternate versions of the tasks. Investigations have demonstrated that CogState is sensitive to relatively modest changes in cognitive function in healthy and impaired individuals. It has been used to differentiate healthy older adults from those with Mild Cognitive Impairment and to study the progression of impairment in MCI relative to healthy control subjects. The variables derived from this instrument include measures of sustained attention, reaction time, working memory, and new learning.

Stop-signal task: This is among the purest measures of inhibitory control, and an enormous amount of experimental data have been accumulated using this procedure. The stop signal task measures inhibition by engaging subjects in a reaction time task and intermittently presenting a signal designated to cease the previously emitted response. It has the advantage of being a relatively simple procedure and a direct measure of this fundamental executive control process. Also, it has been used to demonstrate differences in inhibitory control across development from childhood through old age. A visual version has been programmed in our laboratory using the SuperLab Pro software (Cedrus Corporation) and used in our pilot studies.

California Verbal Learning Test: The California Verbal Learning Test (CVLT) is among the best designed of a number of list learning procedures used to assess memory consolidation ability. It provides data on learning rate or efficiency, interference effects, recall, and recognition memory. Reliability and validity data are adequate and well established. Our data with a middle-aged sample indicated a trend for recall and recognition memory effects on this procedure. In addition, there are normative data for adults across the lifespan, and data indicating that certain CVLT measures can be used with elderly adults to predict familial history of dementia and to identify those at greater risk for progression to dementia.

Verbal Paired Associate Learning: The Verbal Paired Associate Learning task (V-PAL) was developed by the PI and has been used in nonclinical standardization studies and in clinical research. In general, verbal paired associate tasks are very sensitive to the decline observed in cognitive aging and in Alzheimer's disease. As indicated in the preliminary data section, our task (the V-PAL) has shown sensitivity to developmental decline across the adult life span and was particularly sensitive to memory difficulties in the aged. This type of memory task is included in addition to the CVLT because it requires a different type of information encoding, in that it calls for the subject to form novel semantic associations as opposed to acquisition of a word list. The paired associative task may have differential sensitivity as compared with the list-learning task. Alternate, equivalent forms are available for repeated administrations.

IQ estimate: The Peabody Picture Vocabulary Test-III (PPVT) is a multiple-choice measure of receptive vocabulary, which is highly correlated with verbal and full scale IQ. It yields a standard score equivalent with mean of 100 and standard deviation of 15. It will provide a means of characterizing the overall intellectual ability level of the subjects. Lexical knowledge is less vulnerable to early age-related deterioration than other cognitive functions requiring online processing such as executive function ability, attention, and memory, so that estimates of overall ability based on lexical knowledge are relatively accurate representations of pre-decline levels. The PPVT will be administered during the baseline assessment only.

Salivary cortisol: Evening and morning saliva samples are used to measure cortisol levels. Each sample is obtained by placing absorbent material under the tongue at bedtime and upon awakening in the morning. The subjects are instructed to keep the labeled containers and swabs at the bedside and to obtain the samples just before retiring for sleep and as soon as possible after awakening, optimally before getting out of bed. The salivary measure represents a convenient and inexpensive means of evaluating cortisol levels and is quick, painless, and accurate. The morning salivary sample provides the optimal time to detect basal cortisol levels. The addition of the nighttime sample increases the accuracy of the characterization of the circadian cycle. Samples are stored in a freezer until analyzed. The Kallestead Quanticoat radioimmunoassay from Sanofi Diagnostics (Chaska, Minn.) are used to measure cortisol levels based on established techniques.

The salivary samples are obtained the night before and the morning of the baseline and termination evaluations and retrieved from the subjects during their visits to the medical center for the other studies. As noted above, each subject or a member of his or her family is reminded by means of a telephone call on the day before the cortisol sampling as to when and how to obtain the samples in order to augment the previously provided oral and written instructions.

Blood samples: Fasting blood samples are obtained at the baseline and final visits. Blood is drawn in the usual manner from a vein inside of the elbow or the back of the hand. The blood samples include two red top and two green top 7 ml tubes. Serum and plasma are obtained following centrifugation at 2500 g for 20 minutes. Serum is aliquoted into two, 1 ml storage tubes and the remaining in a 5 ml cold storage tube. Plasma is aliquoted in a similar manner. All tubes are stored at −70° C. until analysis.

Seven-day diet diary: We have developed a food record diary adapted from the Women's Health Initiative Four-Day Food Record. Our diet diary covers a period of seven days and serves as a record of all foods and beverages consumed and amounts thereof. In addition, we have added items inquiring about vitamin, mineral, and antioxidant supplements and items designed to record specifically when the study pills are taken. The purpose of this diet record is to provide an estimate as to the total amount of antioxidant intake from food and supplementation beyond that received in the context of the study treatments. It also may help maintain compliance with the study regimen. This instrument is completed by the participants during the week before the baseline visit, the week before the interim visit, and the week before the final visit. This schedule should provide an adequate sampling of the food and supplement intake of the subjects and will limit the burden and inconvenience associated with completing the diary.

### Chromium Supplement and Placebo Regimen

Chromium picolinate: The chromium supplement is provided by Nutrition21, Purchase, N.Y., a major distributor of the chromium picolinate supplement form. A single capsule 1000 mcg dose is administered to subjects receiving the chromium treatment with the morning meal each day for the duration of the intervention. Similar appearing placebo capsules are administered in a similar manner to subjects receiving the placebo treatment.

Randomization and preparation of supplement: The investigators and subjects are blind as to group membership. The randomization schedule is determined before subject assignment. Group assignment is accomplished using the forced-randomization technique of Taves (1974). The subject assignment code is not revealed until completion of the study.

### Data Management, Statistical Analyses, Power, and Sample Size Determination

Each of the primary hypotheses regarding enhancement in cognition and motor function is tested with separate analyses of variance (ANOVA) for each domain of functioning—working memory and secondary memory. Contingent on the degree of within group correlation between the baseline and termination measures, these ANOVA analyses include either covariate control of the baseline measures or will be repeated measures analyses. The former is performed in the case of higher correlations between the baseline and termination measures, while the repeated measures ANOVA is performed if weak associations are observed. Covariance analyses, controlling for baseline effects, have less error variance and greater statistical power, if there exist higher correlations between initial and endpoint measures. For the executive function and memory domains, which are assessed by more than one measure, multivariate analyses are performed. Group is the independent factor and data from the termination evaluations will be the dependent measures. In the case of covariate analyses, the covariate measures for each analysis include demographic variables (age, education, and IQ estimate), mood, and the baseline measure of the dependent variable. Significant main and interaction effects are followed up with univariate between-groups comparisons. Ancillary multiple regression analyses examining the relationships among the outcome measures also is performed.

### Results—Study 1

21 men and women are selected to receive either the chromium picolinate treatment containing 1000 mcg elemental chromium (n=11) or placebo (n=10) for 12 weeks. There is a trend for an age difference between the groups (73 v 69 years; p=0.15), although there is no difference with respect to educational level (15.7 v 15 years), stage and extent of memory impairment (Clinical Dementia Rating sum boxes score, 1.0 v 0.85), or level of mood disturbance (Profile of Mood States total score, 18.4 v 16.9).

At treatment termination, chromium to creatinine ratios are significantly elevated in the supplemented group, F=8.85, p=0.008. No group differences in accuracy of memory on the California Verbal Learning Test are observed. However, a trend for reduced semantic interference is observed. See FIG. 1. Additionally, there is a trend for enhanced motor speed on the Trail-Making task. See FIG. 2. Effect sizes for these differences are in the moderate range.

The trends are corroborated by differences in activation patterns of pre- and post-intervention fMRI scans for individual subjects from the chromium picolinate treatment group and placebo groups. See FIGS. 3A and 3B. Scans for the chromium picolinate treatment group showed increasing activation in left frontal and left parietal cortices in association with increasing working memory load and reduced nonspecific right hemisphere activation. Scans for the placebo-treated subject showed relatively constant activation in both left and right hemispheres

These results support the theory that supplementation with chromium picolinate in older adults with early memory changes can enhance cognitive inhibitory control and motor speed and associated cerebral function.

### Results—Study 2

13 men and women are selected to receive either the chromium picolinate treatment containing 1000 mcg elemental chromium (n=9) or placebo (n=4) for 12 weeks. There is no significant difference between the groups (73 v 72 years), educational level (15.8 v 17.3 years), stage and extent of memory impairment (Clinical Dementia Rating sum boxes score, 0.89 v 0.63), or level of mood disturbance (Profile of Mood States total score, 11 v 4.5).

A neuropsychological protocol is administered and fMRI scans are obtained before and after treatments using a Varian INOVA 4 Tesla scanner. Two runs of BOLD EPI, 30 contiguous 5 mm coronal slices are acquired. Images are obtained while subjects performed the NIH visual-spatial n-back task with 0- and 1-back blocks. There are 20, 1.5 s trials per block; each block repeated 6 times. Image analyses are performed using AFNI and included co-registration, motion correction, blur, normalization, and event-related analysis. Regression analyses includes group, visit, and condition.

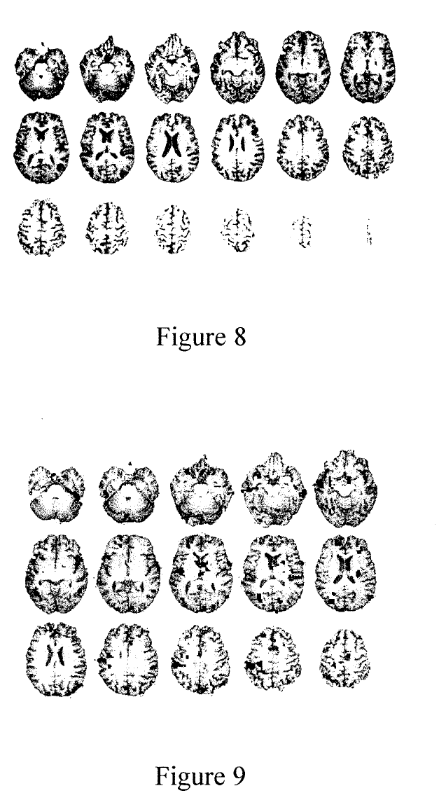

At treatment termination, chromium to creatinine ratios are elevated in the supplemented group, F=8.86, p<0.0001. Activation increases in post-treatment relative to baseline scans for subjects receiving the chromium treatment but not for those receiving placebo. The chromium treated subjects showegreater activation (p=0.005 corrected) relative to placebo treated subjects in right thalamic, right temporal, right posterior parietal, and bilateral frontal regions. See FIG. 8. The difference in activation is not reflected in differential performance on the working memory task, possibly due to the small number of subjects. However, on a measure of memory function (California Verbal Learning Test) administered outside the scanner, the chromium-treated subjects demonstrate reduced intrusion errors relative to the placebo group, suggesting improved executive ability. To explore the implications of increased cerebral activation, a regression analysis is performed, examining the relationship of CVLT free recall intrusions with fMRI cerebral activation. This analysis shows that those with more intrusion errors have greater activation in left prefrontal cortex during the working memory task (p<0.05 corrected). See FIG. 9.

These findings indicate that chromium picolinate supplementation increases brain activation during performance of a resource-intensive task in older adults with early memory decline. In addition, subjects who commit more intrusion errors on a neuropsychological measure of memory have increased activation in left prefrontal cortex. Instrusion errors on memory tasks are thought to index executive disinhibition, which would support the notion that those with greater impairment of inhibitory control allocate greater prefrontal resources while performing the list learning task.

No group differences in accuracy of memory on the California Verbal Learning Test is observed. However, a trend for reduced semantic interference is observed. See FIG. 1. Additionally, there is a trend for enhanced motor

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. A method of treating a cognitive condition comprising:
identifying a mammal suffering from a cognitive condition; and
administering an effective amount of a chromium complex, or pharmaceutically acceptable salt thereof, to said mammal.

2. The method of claim 1, wherein the cognitive condition comprises Alzheimer's disease.

3. The method of claim 1, wherein the cognitive condition comprises mild cognitive impairment.

4. The method of claim 1, wherein the cognitive condition comprises is associated with reduced memory functioning.

5. The method of claim 1, wherein the cognitive condition is associated with reduced brain insulin levels.

6. The method of claim 1, wherein the amount is effective to inhibit cognitive degradation.

7. The method of claim 1, wherein the amount is effective to slow progression of memory impairment.

8. The method of claim 1, wherein said chromium complex is selected from the group consisting of chromium picolinate, chromic tripicolinate, chromium nicotinate, chromic polynicotinate, chromium chloride, chromium histidinate, chromium yeast, chromium nicotinate-glycinate, chromium phenylalanine.

9. The method of claim 1, further comprising administering a cholinesterase inhibitor, memanitine, vitamin E, phospholipid or omega-3 fatty acid to said individual.

10. The method of claim 1, wherein said mammal is a human.

11. A method of treating a cognitive condition comprising:
identifying a mammal at risk of suffering from a cognitive condition; and
administering an effective amount of a chromium complex, or pharmaceutically acceptable salt thereof, to said mammal.

12. The method of claim 11, wherein the mammal has a genetic risk factor comprising a mutated gene.

13. The method of claim 11, wherein the mammal has a family history of the cognitive condition.

14. The method of claim 11, wherein the amount is effective to delay onset of the cognitive condition.

15. The method of claim 11, wherein said chromium complex is selected from the group consisting of chromium picolinate, chromic tripicolinate, chromium nicotinate, chromic polynicotinate, chromium chloride, chromium histidinate, chromium nicotinate-glycinate, chromium phenylalanine, chromium yeast.

16. The method of claim 11, further comprising administering a cholinesterase inhibitor, memanitine, vitamin E, phospholipid or omega-3 fatty acid to said individual.

17. A method of treating memory impairment comprising:
identifying a mammal suffering from age-related memory impairment; and
administering an effective amount of a chromium complex, or pharmaceutically acceptable salt thereof, to said mammal.

18. The method of claim 17, wherein the mammal is at least about 70 years old.

19. The method of claim 17, wherein the amount is effective to inhibit progression of long-term memory impairment.

20. The method of claim 17, wherein the amount is effective to inhibit progression of long-term memory impairment.

21. The method of claim 17, wherein said chromium complex is selected from the group consisting of chromium picolinate, chromic tripicolinate, chromium nicotinate, chromic polynicotinate, chromium chloride, chromium histidinate, chromium nicotinate-glycinate, chromium phenylalanine, chromium yeast.

22. The method of claim 17, further comprising administering a cholinesterase inhibitor, memanitine, vitamin E, phospholipid or omega-3 fatty acid to said individual.

23. A method for supporting cognitive function, comprising:
providing a dietary chromium complex to an individual;
advising the individual that the dietary chromium complex may support cognitive function; and
administering or self-administering the dietary chromium complex to the individual.

24. The method of claim 23, wherein the chromium complex is selected from the group consisting of chromium picolinate, chromic tripicolinate, chromium nicotinate, chromic polynicotinate, chromium chloride, chromium histidinate, chromium nicotinate-glycinate, chromium phenylalanine, chromium yeast.

25. The method of claim 23, further comprising administering a cholinesterase inhibitor, memanitine, vitamin E, phospholipid or omega-3 fatty acid to said individual.

26. The method of claim 9, wherein said method comprises administering a cholinesterase inhibitor to said mammal, and wherein said cholinesterase inhibitor and said chromium complex are present in separate pharmaceutical formulations or nutritional supplements.

27. The method of claim 9, wherein said method comprises administering a cholinesterase inhibitor to said mammal, and wherein said cholinesterase inhibitor and said chromium complex are present in a single pharmaceutical formulation or nutritional supplement.

28. The method of claim 16, wherein said method comprises administering a cholinesterase inhibitor to said mammal, and wherein said cholinesterase inhibitor and said chromium complex are present in separate pharmaceutical formulations or nutritional supplements.

29. The method of claim 16, wherein said method comprises administering a cholinesterase inhibitor to said mammal, and wherein said cholinesterase inhibitor and said chromium complex are present in a single pharmaceutical formulation or nutritional supplement.

30. The method of claim 22, wherein said method comprises administering a cholinesterase inhibitor to said mammal, and wherein said cholinesterase inhibitor and said chromium complex are present in separate pharmaceutical formulations or nutritional supplements.

31. The method of claim 22, wherein said method comprises administering a cholinesterase inhibitor to said mammal, and wherein said cholinesterase inhibitor and said chromium complex are present in a single pharmaceutical formulation or nutritional supplement.

32. The method of claim 25, wherein said method comprises administering a cholinesterase inhibitor to said mammal, and wherein said cholinesterase inhibitor and said chromium complex are present in separate pharmaceutical formulations or nutritional supplements.

33. The method of claim 25, wherein said method comprises administering a cholinesterase inhibitor to said mammal, and wherein said cholinesterase inhibitor and said chromium complex are present in a single pharmaceutical formulation or nutritional supplement.
